(19) United States
(12) Patent Application Publication
(10) Pub. No.: US 2022/0339173 A1
(43) Pub. Date: Oct. 27, 2022

(54) ORAL COMPOSITION FOR IMPROVING MEMORY-LEARNING ABILITY

(71) Applicant: UNITEC FOODS CO., LTD., Tokyo (JP)

(72) Inventors: Momoko ASANO, Tokyo (JP); Hideyoshi CHO, Tokyo (JP)

(73) Assignee: UNITEC FOODS CO., LTD., Tokyo (JP)

(21) Appl. No.: 17/638,939

(22) PCT Filed: Aug. 12, 2020
(86) PCT No.: PCT/JP2020/030641

§ 371 Date: Feb. 28, 2022

(30) Foreign Application Priority Data
Aug. 28, 2019 (JP) 2019-155302

(51) Int. Cl. A61K 31/683; A23L 11/00; A61K 36/484

(52) U.S. Cl. CPC A61K 31/683; A23L 11/03; A61K 36/484; A23K 10/00

(57) ABSTRACT

Provided is a novel functional composition having an excellent beneficial effect on memory-learning ability that can also be utilized in the form of a food or beverage, etc. Phosphatidylinositol is used as an active ingredient of an oral composition for improving memory-learning ability. The phosphatidylinositol is preferably derived from soybeans. This composition can be used suitably in the form of a food or beverage, food additive, pharmaceutical, supplement, animal feed, etc.

### TECHNICAL FIELD

The present invention relates to a functional composition relating to brain function.

### BACKGROUND ART

Phosphatidylinositol (sometimes abbreviated hereinafter as “PI”) is a phospholipid constituting the cell membrane of eukaryotes, including humans. Phosphatidylinositol, like phosphatidylcholine, phosphatidylethanolamine, and other cell membrane-constituting phospholipids, is known to be deeply involved in information transmission function inside and outside the cell. Since the content of phosphatidylinositol and phospholipids related thereto has recently been found to decrease with age, these brain chemicals are thought to play important roles relating to the constitution and functionality of the brain after adulthood.

Patent Document 1 describes the preparation of a phosphatidylinositol-containing material having an increased content of phosphatidylinositol by treating crude phospholipid derived from a natural product such as soybeans by a microbially derived enzyme (phospholipase) to decompose and remove phospholipids other than phosphatidylinositol. The phosphatidylinositol-containing material obtained is described as also being utilizable in the form of a food or beverage, etc.

### RELATED ART DOCUMENTS

### Patent Documents

[Patent Document 1] WO2007/010892 pamphlet

### SUMMARY OF THE INVENTION

### Problems the Invention is Intended to Solve

However, while phosphatidylinositol has been clarified in the past to play various important roles as an endogenous brain chemical, it had not been clarified whether oral administration of phosphatidylinositol has a beneficial effect on brain function.

Therefore, an object of the present invention is to provide a novel functional composition that, by utilizing phosphatidylinositol, has an excellent beneficial effect on memory-learning ability and can also be utilized in the form of a food, beverage, etc.

### Means for Solving the Problems

The present inventors conducted in-depth research to achieve the above goal, discovered that phosphatidylinositol acts to improve memory-learning ability, and perfected the present invention.

Specifically, the present invention, according to a first aspect, provides an oral composition for improving memory-learning ability, characterized by containing phosphatidylinositol as an active ingredient.

In the composition, it is preferable to have an action to raise c-Fos activity in the hippocampus.

In the composition, the phosphatidylinositol is preferably derived from soybeans.

In the composition, it is preferably in the form of a food or beverage, food additive, pharmaceutical, supplement, or animal feed is preferred.

The present invention, according to a second aspect, provides a use of a phosphatidylinositol-containing material to produce an oral composition for improving memory-learning ability.

In the use, the phosphatidylinositol-containing material is preferably derived from soybeans.

In the use, the phosphatidylinositol-containing material is preferably a material obtained by enzymatically treating soybean phospholipids to increase the content of phosphatidylinositol to 20 mass % or higher.

In the use, the oral composition for improving memory-learning ability is preferably in the form of a food or beverage, food additive, pharmaceutical, supplement, or animal feed.

### Effect of the Invention

According to the present invention, a novel functional composition having an excellent beneficial effect on memory-learning ability that can also be utilized in the form of a food or beverage, etc., can be provided by utilizing phosphatidylinositol.

### BRIEF DESCRIPTION OF THE DRAWINGS

FIG. 1 is a chart showing the results obtained by studying changes in the body weight of rats in Test Example 1.

FIG. 2 is a chart showing the results on the required time (time needed from start to finish of a trial) among the evaluations of the eight-arm radial maze test conducted in Test Example 2.

FIG. 3 is a chart showing the results on working memory errors (WME) among the evaluations of the eight-arm radial maze test conducted in Test Example 2.

FIG. 4 is a chart showing the results on the full select count (number of intrusions into an arm from start to finish of a run) among the evaluations of the eight-arm radial maze test in conducted Test Example 2.

FIG. 5 is a chart showing the results on reference memory errors (RME) among the evaluations of the eight-arm radial maze test in conducted Test Example 2.

FIG. 6 is a chart showing the results on latency time in the evaluation of the step-down passive avoidance test conducted in Test Example 3.

FIG. 7 is a chart showing the results on the number of c-Fos+ cells among the immunohistochemical studies conducted in Test Example 4.

FIG. 8 is a chart showing the results on the number of BrdU+ cells among the immunohistochemical studies conducted in Test Example 4.

### BEST MODE FOR CARRYING OUT THE INVENTION

A composition according to the present invention is one containing phosphatidylinositol.

The phosphatidylinositol used in the present invention may be derived from a natural product or may be synthetic. Derivation from a natural product is preferred for provision in the form of a food or beverage, etc. For example, in Patent Document 1 (WO2007/010892 pamphlet) mentioned above, the preparation of a phosphatidylinositol-containing material having an increased content of phosphatidylinositol by treating crude phospholipids derived from a natural product such as soybeans with a microbially derived enzyme (phospholipase) to decompose and remove phospholipids other than phosphatidylinositol is described. A phosphatidylinositol-containing material derived from a natural product can be prepared by such a known method or a method similar thereto and used in a composition according to the present invention. Alternatively, a commercial phosphatidylinositol-containing material may be utilized. Furthermore, “phosphatidylinositol” is a general term for phospholipids having an inositol ring; for example, some seven types are known in nature. The phosphatidylinositol that can be used is not particularly restricted as to type, and multiple types may be used in mixture or combination. Also, quantitative identification of phosphatidylinositol can be handled as the total amount of the one or several types detectable.

When using a phosphatidylinositol-containing material derived from a natural product, its origin, etc., is not particularly restricted. For example, phospholipids derived from soybeans, phospholipids derived from corn, phospholipids derived from sunflower, phospholipids derived from Brassica rapa, bovine phospholipids, etc., can be used, but phospholipids derived from soybeans and sunflower are preferred, and phospholipids derived from soybeans are more preferred, in terms of a stable supply of raw material phospholipids.

When using a phosphatidylinositol-containing material prepared from a natural product such as soybeans, it is preferable to use a material having a phosphatidylinositol content (purity) increased, for example, to 20 mass % or higher, more preferably to 30 mass % or higher, even more preferably to 40 mass % or higher, and especially preferably to 50 mass % or higher. Alternatively, it is preferable to use a material in which the total phospholipid content excluding phosphatidylinositol is, for example, 40 mass % or less, more preferably 30 mass % or less, and even more preferably 20 mass % or less.

Here, the phospholipid content can be measured by a method known to one skilled in the art. In particular, the measurement method desirably involves quantitative analysis of the phospholipid composition by high-performance liquid chromatography stipulated as a measurement method established by the Japan Oil Chemists' Society. For example, specific phospholipids such as phosphatidylinositol, phosphatidylcholine, phosphatidylserine, phosphatidylethanolamine, phosphotidylglycerol, and phosphatidic acid can be measured by HPLC quantitative analysis or the like using standard products of known concentrations thereof as index samples. In recent years, it has also been possible to conduct quantitative analysis of phospholipids using ³¹P-NMR.

The composition according to the present invention is used to improve memory-learning ability.

Here, the phrase “to improve memory-learning ability” specifically can be improvement of brain functionality via the hippocampus. Specifically, memory-learning ability is known to be controlled in the hippocampus of the brain. The hippocampus is one brain region important for memory-learning in humans, and is known to be an organ essential for memory and learning because serious impairment of memory function occurs when the hippocampus is impaired. The brain can store new experiences as memories through the hippocampus throughout a lifetime. As for new memories stored in the hippocampus, old memories are transferred to the cerebral cortex by the action of neurogenesis, and new memories can be stored in the hippocampus without saturating the memory capacity of the hippocampus. Information is said to be organized over about one month in the hippocampus, which is therefore thought to control memory management. Also, after acquiring a memory, the work of the hippocampus is required to recall the memory. The hippocampus is also known to have functions related to spatial recognition and navigation.

New nerve cells are produced continuously in the hippocampus even in adults who have completed brain development; this is called “neurogenesis.” It is known that neural stem cells are present in the hippocampus even after the brain has been completed and that new nerve cells are produced continuously, but the magnitude of production decreases with age. Neurogenesis is also known to be reduced by a lifestyle that does not use brain function, even unassociated with aging.

Therefore, activation of the work of the hippocampus means that, in certain cases, the ability to acquire, organize, and retain new memories, that is, the memory faculty, is enhanced, and the ability to recall memories is enhanced. In other cases, activation of the work of the hippocampus can be said to have the effect of preventing decreases in memory-learning ability which declines with age. Also, in another case, the function of judging one's situation based on time, place, person, etc. is called orientation, and this orientation can be improved. More specifically, activation of the work of the hippocampus can be explained by effects such as “facilitated input and output of memories,” “improved spatial learning ability,” “facilitated comprehension of image and position information,” etc. Looking at each generation, for growing children, activation of the work of the hippocampus can be explained by effects such as “quick learning of words,” “improvement of vocabulary, reading comprehension, and native language ability,” “strong math skills,” “not getting lost,” etc. In the learning years such as high school and college, activation of the work of the hippocampus can be explained by effects such as “strong ability to focus,” “improved memorization ability,” “facilitated learning of a foreign language,” “answering quickly on tests,” etc. In middle age, activation of the work of the hippocampus can be explained by effects such as “improved work efficiency,” “answering inquiries quickly,” “improved map recognition ability,” etc. In old age, activation of the work of the hippocampus can be explained by effects such as “reduced forgetfulness,” “improved ability to remember,” “quicker memory recall,” “not getting lost,” “losing fewer things,” “finding lost items quickly,” etc.

Therefore, the composition according to the present invention is one intended to obtain improvement of these brain functionalities. Furthermore, “improvement” means not only to make the current situation better but also to maintain the current state and prevent deterioration thereof.

The composition according to the present invention can be used suitably, for example, in healthy persons such as growing children, middle-aged members of society, and middle-aged and older persons. More specifically, the composition can be used more suitably in healthy persons 40 years and older. Also, the composition can be used suitably not only in humans but also in animals other than humans that have memory-learning ability as described above, such as dogs, cats, and other pet animals.

The usage form of the composition according to the present invention may be such that phosphatidylinositol, the active ingredient for improving memory-learning ability as described above, is ingested orally; for example, phosphatidylinositol may be contained in a predetermined form to be ingested orally. The phosphatidylinositol content in the composition may be adjusted suitably from the viewpoint of ensuring the desired dose and is not particularly restricted. For example, the content may be within a range of 0.001 to 99.99 mass %, within a range of 0.01 to 80 mass %, within a range of 0.1 to 50 mass %, within a range of 1 to 30 mass %, or within a range of 3 to 20 mass %.

The composition according to the present invention may be prepared, for example, in the form of a food or beverage, food additive, pharmaceutical, supplement, animal feed, etc., together with appropriate additives, pharmaceutical materials, etc.

Typical examples of food and beverage forms include jellies, puddings, gummies, gums, chocolates, cookies, candies, and other such confections; breads; cooking oils; mayonnaise, dressing, butter, cream, margarine, and other such fat/oil foods; ketchup, sauces, and other such seasonings; milk, yoghurt, cheese, and other such dairy products; udon, soba, ramen, pasta, yakisoba, kishimen, somen, hiyamugi, rice noodles, and other such noodles; miso soup, corn soup, consommé, and other such soups; tea, carbonated beverages, lactic acid beverages, sports drinks, and other such beverages; liquid diets, sprinkles, etc.

Typical examples of supplement and pharmaceutical forms include powders, granules, soft capsules, hard capsules, tablets, chewable tablets, quick-dissolving tablets, syrups, liquids, etc.

Also, in certain cases, phosphatidylinositol may take on the form of liposomes. That is, phosphatidylinositol can be a constituent lipid of a lipid bilayer membrane of liposomes. Absorption from the intestine, etc., when orally ingested is improved by making liposomes. In addition to being in liposomal form, phosphatidylinositol may be emulsified, microemulsified, as a means for improving the absorption rate.

The preferred dosage of the composition according to the present invention can be determined, as appropriate, depending on the age and health status of the person or animal being administered, the duration of administration, frequency of administration, etc. To give a general dosage example, phosphatidylinositol can be administered in a dose of 2.0 to 3000.0 mg/kg (body weight)/day. Also, phosphatidylinositol may be used by continuous ingestion over a predetermined period of time; for example, phosphatidylinositol may be used so as to be ingested continuously over one month or longer. Furthermore, safety was confirmed in in vitro mutagenicity studies and single-dose and 90-day repeated-dose studies in rats using materials containing a high phosphatidylinositol content of 50 mass % or more. The LD50 by single dose in rats is reported to be at least 2000 mg/kg (body weight)/day (Honda et al., The Journal of Toxicological Sciences, 2009, vol. 34, no. 3, p. 265-280).

### Examples

The present invention is explained more specifically below through examples, but these examples do not limit the scope of the present invention.

1. Experimental Materials

A soybean-derived material “Soy Brain (registered trademark) PI 50” (Unitec Foods Co., Ltd.), which has constitutes shown in table below, was used as the phosphatidylinositol-containing material.

TABLE 1

| | Content in material (mass %) |
|---|---|
| Phosphatidylinositol | 55.7 |
| Phosphatidylcholine | 2.5 |
| Phosphatidylethanolamine | 4.8 |
| Phosphatidic acid | 1.8 |
| Total | 64.8 |

2. Animals Used and Rearing Environment

Male Wistar rats (body weight: approximately 220-250 g) were used as animals to eliminate the effects of hormones. The rats were given commercial feed (CE-2, Japan CLEA) and water ad libitum and reared in an animal room controlled to 12 hours each of light and dark, at a temperature of 25.0° C. and humidity of 55±5%.

3. Administration of Test Substance

In the control group, distilled water and in the PI groups, the above soybean-derived phosphatidylinositol-containing material dissolved in distilled water in amounts of 3 mg/kg, 10 mg/kg, or 30 mg/kg were administered directly to the stomach in a volume of 1 mL/once a day using an oral administration device (each group, n=10). The duration of administration was set at one month each. In this study, 100 mg/kg of BrdU (bromodeoxyuridine) was administered intraperitoneally (IP injection) to the rats once a day from 30 minutes to one hour before oral administration of PI. BrdU is known as a marker for detecting proliferating cells and measuring the degree of DNA synthesis.

4. Statistical Processing

Statistical processing of the results of the study calculated the mean±standard deviation (S.D.) of the data of the eight-arm radial maze test and the step-down passive avoidance test, which are behavioral methods. Test data of the immunohistochemical studies were shown as the mean±standard error (S.E.). One-way ANOVA was used for differences between groups, and multiple comparisons were performed using the Scheffe test as a post-test. Furthermore, the significance level was set at 5% (p<0.05).

### [Test Example 1] (Changes in Body Weight)

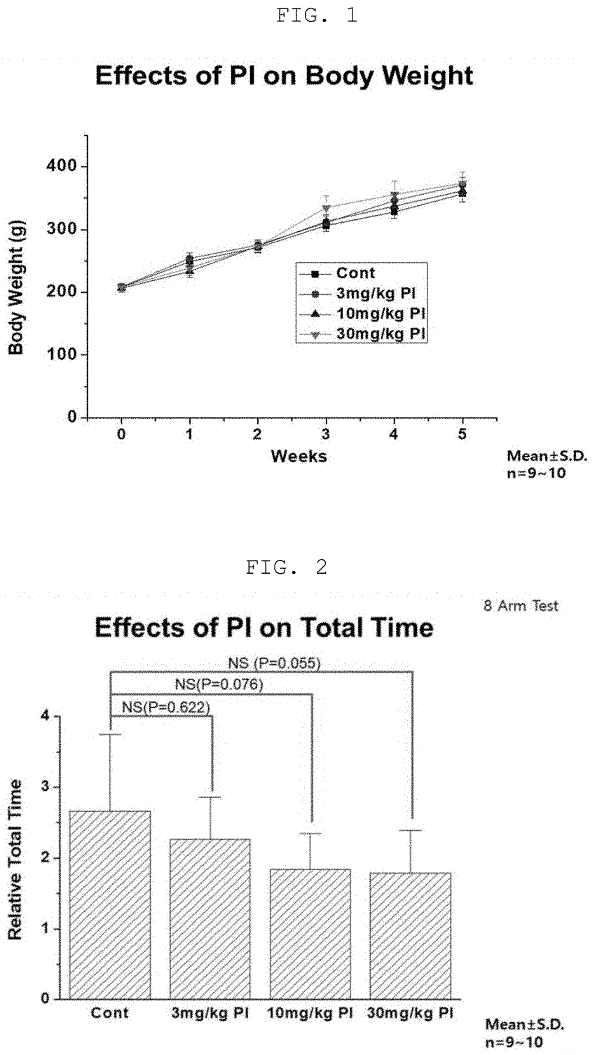

The body weights were recorded weekly, taking the day before administration of the test substance as day 0, and changes therein were investigated. As a result, as shown in FIG. 1, there was a weight gain of about 200 g in the control group and each PI group during the administration period, and no particular effects of PI administration could be seen.

### [Test Example 2](Eight-Arm Radial Maze Test)

An eight-arm radial maze test, known as a method for evaluating spatial learning-working memory, was carried out to evaluate the memory-learning ability of rats. An acrylic elevated directional maze comprising an octagonal central platform with a side wall height of 18 cm and a diameter of 28 cm at a position 60 cm above the floor and eight arms 43 cm long×11 cm wide connected to the central platform was created as a maze device configuration for this purpose. Also, to make the effects of the outside scenery constant, the four sides were enclosed by gray plastic plates up to a height of 90 cm from the floor and square, circle, star, and triangle patterns were drawn on each side so as to be visible to the rat. In addition, a fixed-point camera was installed at a position 190 cm above the floor directly above the central platform for observation.

After a one-week adaptation period for acclimation to the device, prior learning relating to water searching behavior was performed utilizing the eight-arm radial maze test as described below three days before beginning administration of the test substance. Oral administration continued for one month thereafter, and the main test utilizing the same eight-arm radial maze test was performed on the final day of administration. To install water, white lids 3 cm in diameter and 1 cm deep were installed as containers at the tips of all eight arms, and the test was begun by putting 50 μL of water in only four of the eight arms and not putting water in the remaining four containers. For the test, water restriction was implemented for 24 hours before the start, and water searching behavior was observed and analyzed. The required time, working memory errors (WME), full select count, and reference memory errors (RME) were measured as indicators of spatial cognitive function. Furthermore, a maximum of 10 minutes was set for the required time.
Required time: time needed from start to end of run (finished by drinking water)
Working memory errors (WME): number of times an arm already entered was re-entered
Reference memory errors (RME): number of times an arm with no water was selected
Full select count: number of intrusions into arms from start to end of run

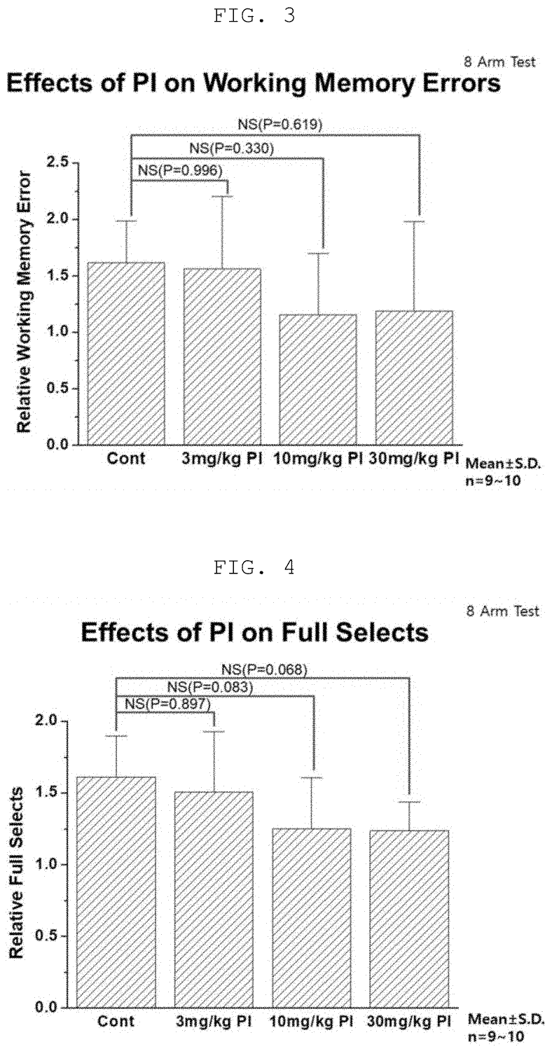

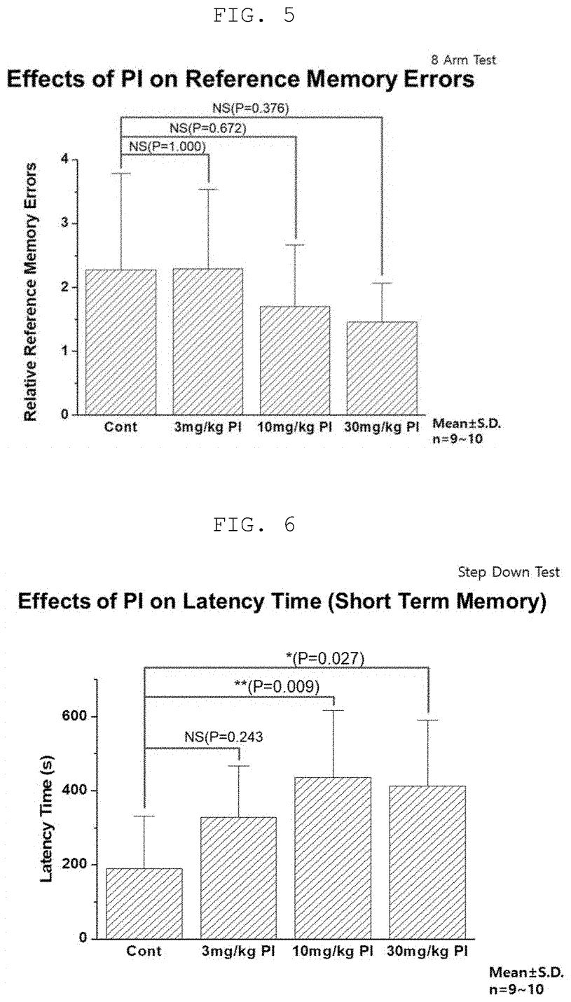

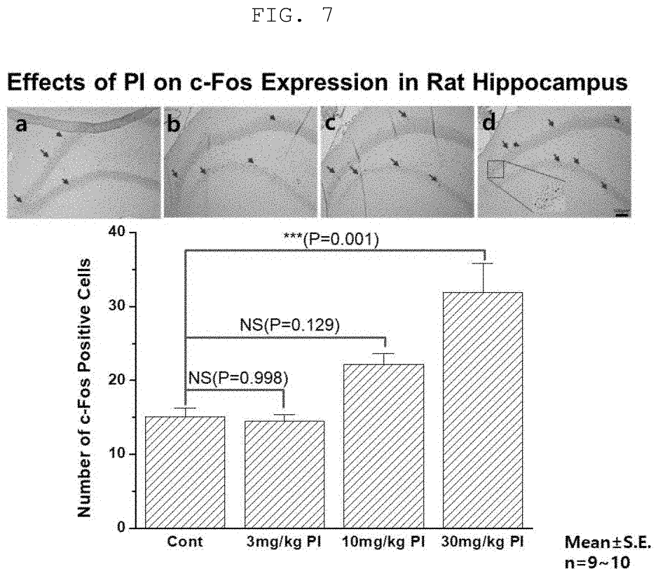

As a result, as shown in FIGS. 2-5, all indices of the required time, working memory error (WME), full select count, and reference memory error (RME) tended to decrease, dependently on the PI dose, in the PI groups in comparison to the control group.

### [Test Example 3] (Step-Down Passive Avoidance Test)

After performing the eight-arm radial maze test after one month of administration of the test substance, a passive avoidance test (step-down type), known as a method for evaluating short-term memory, was carried out. In the test, a 50 cm×25 cm×30 cm transparent acrylic box was used. The right side of this box was equipped with a 5 cm×12 cm×25 cm long platform, and a series of stainless steel rods (diameter 2 mm) were placed at 2 mm intervals on the left side. In preliminary learning, a rat was placed on the 5 cm high platform. After a step-down waiting time of 20 seconds, if the rat descended from the platform to the stainless steel rods below and approximately five seconds elapsed, a 30 V electrical stimulation was applied. When the rat that had received the stimulation escaped to the platform, the rat was removed from the box. The main test was conducted 24 hours after preliminary learning. In this test, the rat was again placed on the platform, and the time until descending completely from the platform to the stainless steel rods below was measured as the latency time. In this case, electrical stimulation was not applied to the stainless steel rods even if the rat descended to the stainless steel rods below.

As a result, as shown in FIG. 6, a significant increase in latency time was found in the PI groups in comparison to the control group (P<0.05 in both the 10 mg/kg group and 30 mg/kg group).

### [Test Example 4] (Immunohistochemical Studies)

After the eight-arm radial maze test and the step-down passive avoidance test had been completed, the abdomen was incised after inducing anesthesia by pentobarbital (10 mg/kg, IP). Brain specimens were prepared after substituting the systemic blood by phosphate buffered saline (PBS). After immersing the brain tissue obtained from the rat in 4% paraformaldehyde (PFA) and fixing the tissue, the tissue was embedded in paraffin and cut in a thickness of 6 μm using a microtome, and hippocampal tissue specimens were prepared.

These sliced specimens were immunostained by anti-c-Fos antibody (Santa Cruz Inc.) or anti-BrdU antibody (Abcam Inc.) by the usual method. Subsequently, 20× photographs were taken by a digital camera for a microscope (BX51, Olympus Inc.), and the number of positive cells within a 200 μm×200 μm field was measured using an imaging counter for a microscope (Cell Sens Dimension Ver. 7, Olympus Inc.).

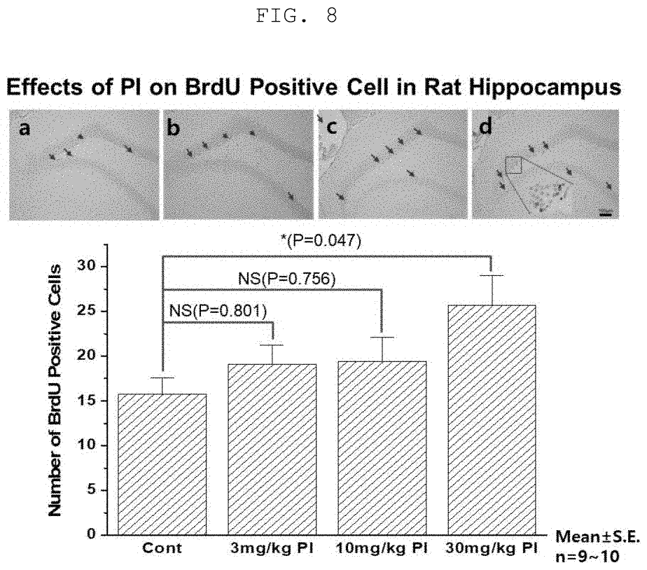

FIG. 7 shows the results of immunostaining by anti-c-Fos antibody.

As shown in FIG. 7, the number of c-Fos+ cells in the hippocampal portion of the brain was found to be significantly increased (p<0.001 in the 30 mg/kg group) in the PI groups in comparison to the control group.

FIG. 8 shows the results of immunostaining by anti-BrdU antibody.

As shown in FIG. 8, the number of BrdU+ cells in the hippocampal portion of the brain was found to be significantly increased (p<0.05 in the 30 mg/kg group) in the PI groups in comparison to the control group.

c-Fos is generally thought to act as a transcription factor that activates other genes; various cascade reactions continue after c-Fos has been expressed, phosphorylation occurs, and cellular responses change. In addition, BrdU is a nucleic acid precursor that, when DNA synthesis becomes active, is taken up into nascent DNA and is a cell proliferation marker.

Thus, based on these immunostaining results, it is thought that nerve cell activation and cell proliferation occur in a portion of the hippocampus in the PI groups, that is, that neurogenesis is activated, in comparison to the control group. This is a phenomenon not previously reported even for other phospholipids. For example, with phosphatidylserine, one of the phospholipids, it is reported that changes in c-Fos activity in the brain region are found in the paraventricular nucleus, but that there are no changes in c-Fos activity in the hippocampus (Phosphatidylserine Research Group, Bulletin Vol. 3, No. 1, Nov. 2005, 10-15). Therefore, the function of improving c-Fos activity in the hippocampus is found for the first time only in phosphatidylinositol among the phospholipids. Phosphatidylinositol is known to be degraded into inositol by phospholipase C present in the body. Inositol is a substance that acts as an osmolyte that maintains osmotic pressure in the body and works to supply nutrients to nerve cells. Although the pharmacokinetics after oral ingestion are not clear, there is thought to be a possibility based on the present results that both phosphatidylinositol and the degraded inositol acts on neurogenesis.

In the past, neurogenesis was thought to be largely unique to the development process, and the amount of neurogenesis was thought to decrease as one grew up. However, the phenomenon of the birth of new nerve cells in the brain has been confirmed, even after adulthood, in the hippocampus and subventricular zone. Although the definite role of neurogenesis has not been elucidated, it is thought based on the above results that PI administration activated neurogenesis in the hippocampus, which led to a beneficial effect on memory-learning ability.

### [Production Example 1] (Jelly)

A quantity of 80 g of sugar, 0.3 g of sweetener, 5.5 g of a gelling agent (“Unigum A-25,” Unitec Foods Co., Ltd.), and 10 g of the soybean-derived material “Soy Brain (registered trademark) PI 50” (Unitec Foods Co., Ltd.) used in Test Example 1-4 were mixed, dispersed by stirring in 800 g of water, and heated to dissolve. A quantity of 10 g of grapefruit juice with pulp, 3 g of citric acid, and 1.2 g of sodium citrate dissolved in 100 g of hot water was mixed with the dissolved solution. After adjusting the weight to a total of 1000 g, 2.5 g of grapefruit flavor and honey flavor was added. Jelly containers were filled with 150 g each of the resultant mixture and sealed. Each container was subsequently sterilized and cooled to produce jelly. The jelly obtained had moderate grapefruit acidity and a fresh flavor.

### [Production Example 2] (Soymilk Pudding)

A quantity of 30 g of dextrin, 10 g of modified starch, 6 g of a gelling agent (“Unigum A-25,” Unitech Foods Co., Ltd.), 10 g of the soybean-derived material “Soy Brain (registered trademark) PI 50” (Unitec Foods Co., Ltd.) used in Test Examples 1-4, 2 g of an emulsifier, and 1.5 g of a fermented cellulose preparation were mixed, dispersed by stirring in 500 g of prepared soymilk and 400 g of water, and heated to dissolve. After stirring the dissolved solution for ten minutes at 10,000 rpm using a homogenizer and homogenizing, the weight was adjusted to 1000 g, and containers were filled with 300 g portions. After filling, the containers were sterilized by retort processing and cooled to produce pudding. This pudding had a smooth texture and a good flavor of soymilk.

### [Production Example 3] (Powdered Soup)

After preparing a mixture (total of 100 mass parts) of 30 mass parts of corn powder, 20 mass parts of skim milk powder, 21.7 mass parts of starch, 9 mass parts of onion powder, 15 mass parts of the soybean-derived material “Soy Brain (registered trademark) PI 50” (Unitec Foods Co., Ltd.) used in Test Examples 1-4, 0.3 mass part of an emulsifier, 3 mass parts of salt, and 1 mass part of powdered bouillon as a blend composition, the mixture was processed into granules using a granulator. The process was completed by filling aluminum three-sided sticks with 20 g each. When one packet of the powdered soup produced in this way was dissolved in 200 g of hot water, the soup had appropriate saltiness and an umami taste.

### [Production Example 4] (Tablets for Memory Maintenance Supplement)

After preparing 44.9 mass parts of glucose, 10 mass parts of the soybean-derived material “Soy Brain (registered trademark) PI 50” (Unitec Foods Co., Ltd.) used in Test Examples 1-4, 10 mass parts of crystalline cellulose, 30 mass parts of creatine, 5 mass parts of gingko biloba extract powder, and 0.1 mass part of flavoring (total of 100 mass parts) as a blend composition, the mixture was processed into tablets using a tableting machine. There was no problem with processability, and the tablets obtained were very suitable as an easy-to-take supplement.

### [Production Example 5] (Chocolate for Learning Supplement)

A chocolate base comprising 35.4 mass parts of sugar, 20 mass parts of chocolate liquor, 20 mass parts of powdered whole milk, 12 mass parts of cocoa butter, 10 mass parts of vegetable oil, 0.8 mass part of an emulsifier, 0.2 mass part of flavoring, 1 mass part of the soybean-derived material “Soy Brain (registered trademark) PI 50” (Unitec Foods Co., Ltd.), and 0.6 mass part of GABA powder (total of 100 mass parts) as a blend composition was prepared. This chocolate base was warmed by a tempering machine, filled into a molding machine, cooled and solidified, and removed from the molds. The chocolates obtained were packaged, and phosphatidylinositol-blended chocolates were produced. The chocolate flavor of the chocolates obtained was not harmed by phosphatidylinositol addition, and the chocolates were very easy to take even as a snack for children.

1. A method for improving memory-learning ability, comprising administering to a subject phosphatidylinositol as an active ingredient.

2. The method according to claim 1, wherein the subject administered with the phosphatidylinositol has an effect to raise c-Fos activity in the hippocampus.

3. The method according to claim 1, wherein the phosphatidylinositol is in the form of a soybeans-derived composition.

4. The method according to claim 1, wherein the phosphatidylinositol is in the form of a food or beverage, food additive, pharmaceutical, supplement, or animal feed.

5-8. (canceled)
